(19) United States
(12) Patent Application Publication
(10) Pub. No.: US 2015/0119749 A1
(43) Pub. Date: Apr. 30, 2015

(54) SYSTEMS AND METHODS FOR MONITORING FERTILITY USING A PORTABLE ELECTRONIC DEVICE

(71) Applicant: OVATEMP, LLC, Newton, MA (US)

(72) Inventors: Daniel Graf, Boston, MA (US); Ana Mayer, Boston, MA (US); Akshay Kolte, Wellesley, MA (US); Peter Wrisley, Hollis, NH (US)

(21) Appl. No.: 14/396,944

(22) PCT Filed: Mar. 15, 2013
(86) PCT No.: PCT/US13/31923

§ 371 Date: Oct. 24, 2014

Related U.S. Application Data
(60) Provisional application No. 61/639,569, filed on Apr. 27, 2012.

(51) Int. Cl. A61B 5/01; A61B 5/00

(52) U.S. Cl. CPC A61B 5/01; A61B 5/4331
USPC 600/549

(57) ABSTRACT

Devices, systems, and methods are disclosed herein for monitoring a woman's fertility status by tracking basal body temperature and one or more primary indicia of fertility. In accordance with certain aspects of the present teachings, an integrated software application on the user's personal, portable electronic device enables the user to quickly and consistently track various indicia of fertility in order to successfully practice the Fertility Awareness Method. Moreover, the integrated application can provide accurate fertility determinations and relevant information for practicing the method so as to improve the likelihood for conception or contraception.

### RELATED APPLICATIONS

This application claims the benefit of priority of U.S. Provisional Application Ser. No. 61/639,359, filed on Apr. 27, 2012, the contents of which are hereby incorporated by reference in their entirety.

### FIELD OF THE INVENTION

The present invention relates generally to the field of contraception and family planning. Particularly, the present invention relates to devices, systems, and methods for determining a woman's fertility status based on the user's basal body temperature and at least one other indicia of fertility. In various aspects, the systems can enable the user to practice the Fertility Awareness Method using an integrated software application on a portable electronic device and/or provide recommendations to the user for practicing this method.

### BACKGROUND OF THE INVENTION

A normal woman's menstrual cycle generally has three phases, namely: 1) the menses phase, 2) the fertile phase, and 3) the infertile phase. The fertility of the woman, and the likelihood of conception, varies during these phases. For example, during the infertile phase, which typically lasts from ten to twelve days, the woman is completely infertile. During the menses phase, which typically lasts about one week, the woman experiences menstrual bleeding and is unlikely to become pregnant. Following is the fertile phase, during which the woman discharges cervical mucus and ovulates and can be in the state of greatest possible fertility.

Various calendar-based methods have been developed in order to determine the woman's fertility status so as to avoid pregnancy (i.e., as contraception) or to achieve pregnancy. Though calendar-based methods of identifying the fertility status have been widely practiced for thousands of years, the reliability of these methods generally rely on the user's self-evaluation and discipline in tracking menstrual signs. For example, the “rhythm method” statistically predicts a woman's fertile window based solely on the length of the menstrual cycle as determined from the woman's observation of the first day of menstrual bleeding. However, the widely-accepted (and erroneous) formula that every woman's menstrual cycle is 28 days with ovulation occurring on the fourteenth day does not truly track the body's natural functions to determine when a woman is fertile, and thus, often leads to incorrect determinations. It has been estimated that if the rules of the rhythm method are strictly followed, the annual contraceptive success rate is only approximately 13 percent.

Scientific knowledge gained over the past century has greatly increased the accuracy of various calendar-based methods to determine a woman's fertility. By way of example, the Fertility Awareness Method (FAM) consists of monitoring several indicia of the female body throughout the menstrual cycle in order to predict future ovulation, confirm if ovulation has occurred, predict future menstruation, avoid pregnancy, and/or time intercourse appropriately in order to achieve pregnancy. Various primary indicia of the body's natural and normal functioning utilized by FAM to predict ovulation include the woman's basal body temperature, characteristics of the woman's cervical fluid, and/or cervical position. Ideally, these indicia are observed at the same time every day (e.g., upon waking) and recorded such that the data can then be plotted on a chart to help determine fertile and infertile days.

It has been estimated that if the rules of the Fertility Awareness Method are followed precisely, the annual contraceptive success rate is as high as approximately 97 percent. However, because the Fertility Awareness Method utilizes more information so to better determine the woman's fertile period, the method also requires, consistent self-evaluation as well as diligent (and sometimes time-consuming) recording. Moreover, faulty judgments regarding disturbances in waking temperatures, fever or illness, consistency of cervical fluid, and other specific circumstances that play an important part in determining fertility status using the Fertility Awareness Method can lead to errors in reporting and/or inaccurate determinations.

Accordingly, there remains a need to provide a more effective methods and systems of monitoring fertility.

### SUMMARY OF THE INVENTION

In accordance with certain aspects of the present teachings, systems and methods are provided that enable a woman to quickly, accurately, and consistently chart primary and secondary menstrual cycle signs to ensure maximum effectiveness of calendar-based methods of tracking fertility and decrease frustrations caused by unwanted pregnancies or difficulty conceiving. In various aspects, the methods and systems can enable the user to practice the Fertility Awareness Method and/or provide the user recommendations for practicing the same.

In various aspects, a system for monitoring the fertility of a woman is provided that comprises a thermometer configured to acquire basal body temperature data of a user and transmit the basal body temperature data to an electronic device of the user. The electronic device can include a digital data processor configured to receive the basal body temperature data as well as receive input from the user of one or more primary indicia of fertility. The digital data processor can further determine the fertility status of the user based on the basal body temperature data and the primary indicia input data and provide a notification to the user of her fertility status.

The electronic device can be any variety of electronic device including one of a cellular phone, a smartphone, a personal data assistant, a tablet computer, a laptop computer, and a personal computer, all by way of non-limiting example. Further, the portable electronic device can in some embodiments comprise an integrated software application configured to perform various aspects of the present teachings. In various aspects, the digital data processor can be operable to run an integrated application for interfacing with the thermometer, for allowing the user to input one or more of the primary indicia of fertility, for determining the fertility status of the woman, and/or for notifying the user of her fertility status. In some embodiments, the integrated software application can collect and compile indicia used to track fertility and/or display the user's fertility status. Additionally or alternatively, in some aspects, the digital data processor can be further configured to prompt the user for input information regarding one or more of basal body temperature data and the primary indicia of fertility. In some aspects, the digital data processor can be configured to access a calendar function of the portable electronic device (e.g., a native calendar function such as iCal or an Outlook calendar) and display the fertility status thereon. In various aspects, the notification of the woman's fertility status can be one of an e-mail, a text message, a voicemail, a push notification, and a pop-up notification delivered to the portable electronic device. In various embodiments, a method of monitoring fertility in women is provided that includes receiving basal body temperature data of a user of a portable electronic device, the basal body temperature data being generated by a thermometer in communication with the portable electronic device. The method can also include receiving input data from the user of one or more primary indicia of fertility selected from the group consisting of period data, cervical fluid data, cervical softness data, cervical height data, cervical opening data, and cervical sensation data. The method can further include determining a fertility status of the user based on the basal body temperature data and the primary indicia input data and providing a notification to the user of her fertility status. By way of example, the portable electronic device can comprise an integrated software application configured to perform various aspects of the present teachings. By way of non-limiting example, the digital data processor can be operable to run an integrated application for interfacing with the temperature probe, for allowing the user to input one or more of the primary indicia of fertility, for determining the fertility status of the woman, and/or for notifying the user of her fertility status.

In some aspects, the fertility status can be determined by the digital data processor of the portable electronic device. Alternatively, in some aspects, the fertility status can be determined by a remote digital data processor after the basal body temperature data and the primary indicia input data have been transmitted from the portable electronic device to the remote digital data processor.

In various aspects, the method can include acquiring the basal body temperature data of the user using the thermometer. In related aspects, the basal body temperature data can be transmitted from the thermometer to the portable electronic device. Can we add that this is a non excluding example in case in the future we come up with a button in the thermometer to input the ferning in saliva?

In various aspects, the notification can indicate that the user is fertile or not fertile. The notification can be delivered to the user in a variety of manners. By way of example, the notification can be provided by outputting the fertility status to a calendar of the user. In some aspects, the calendar can be displayed by the portable electronic device. In some embodiments, the notification comprises one of an e-mail, a text message, a voicemail, a push notification, and a pop-up notification.

In some aspects of the present teachings, the user can be prompted for input information. For example, the user can be prompted to input information regarding one or more of basal body temperature data and the primary indicia of fertility. In related aspects, the processor can access a user's alarm clock on the portable electronic device and can prompt the user for input information (e.g., a basal body temperature reading) upon being woken by the alarm clock.

In various embodiments, the method can also include receiving input data from the user of one or more secondary indicia of fertility selected from the group consisting of ovulation predictor kits, pregnancy tests, medications, intercourse, ferning, breast fullness and/or tenderness, and disturbances.

In various aspects, the method can further include providing to the user via the portable electronic device at least one of dietary and exercise information related to said fertility status.

In various embodiments, a portable electronic device for monitoring the fertility status of a woman is provided comprising communications circuitry configured to interface with a thermometer for acquiring basal body temperature data of a user, the thermometer having a transmitter for transmitting said basal body temperature data. The portable electronic device can also include an input component for receiving data from the user of one or more primary indicia of fertility. A processor can determine the fertility status of the user based on the basal body temperature data and the primary indicia input data. The device can also include an output component for notifying the user of her fertility status.

In various embodiments, machine-readable media comprising machine-readable instructions recorded thereon are provided for receiving basal body temperature data of a user of a portable electronic device, the basal body temperature data being generated by a thermometer in communication with the portable electronic device, receiving input data from the user of one or more primary indicia of fertility, determining a fertility status of the user based on the basal body temperature data and the primary indicia input data, and providing a notification to the user of her fertility status.

These and other features of the applicants' teachings are set forth herein.

### BRIEF DESCRIPTION OF THE DRAWINGS

The skilled person in the art will understand that the drawings, described below, are for illustration purposes only. The drawings are not intended to limit the scope of the applicants' teachings in any way.

FIG. 1, in schematic diagram, illustrates an exemplary system for monitoring fertility in accordance with various aspects of the applicants' present teaching.

FIG. 2, in schematic diagram, illustrates an exemplary thermometer for use in the system of FIG. 1 and in accordance with various aspects of the applicants' teachings.

FIG. 3, in schematic diagram, illustrates an exemplary electronic device for use in the system of FIG. 1 and in accordance with various aspects of the applicants' teachings.

FIG. 4, in schematic diagram, illustrates an exemplary method of using the system of FIG. 1 for monitoring the fertility of a woman in accordance with various aspects of the applicants' teachings.

FIG. 5, in schematic diagram, illustrates an exemplary display for the electronic device of FIG. 1, the display providing functions available for a user to track indicia of fertility in accordance with various aspects of the applicants' teachings.

FIG. 6, in schematic diagram, illustrates an exemplary display on the electronic device of FIG. 1, the display tracking various indicia of fertility when using the Fertility Awareness Method in accordance with various aspects of the applicants' teachings.

### DETAILED DESCRIPTION

Certain exemplary embodiments will now be described to provide an overall understanding of the principles of the structure, function, and use of the systems and devices disclosed herein. One or more examples of these embodiments are illustrated in the accompanying drawings, which are not necessarily to scale. Those skilled in the art will appreciate that the systems and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Devices, systems, and methods are disclosed herein that provide for monitoring a woman's fertility status by tracking basal body temperature and one or more primary indicia of fertility. In accordance with certain aspects of the present teachings, a more effective determination of fertility status can be performed by utilizing an integrated software application on the user's personal, portable electronic device. In some aspects, the integrated application can allow the user to quickly and consistently track various indicia of fertility such as basal body temperature in order to successfully practice the Fertility Awareness Method. Moreover, the integrated application can provide accurate fertility determinations and relevant information for improving the chances for conception or contraception.

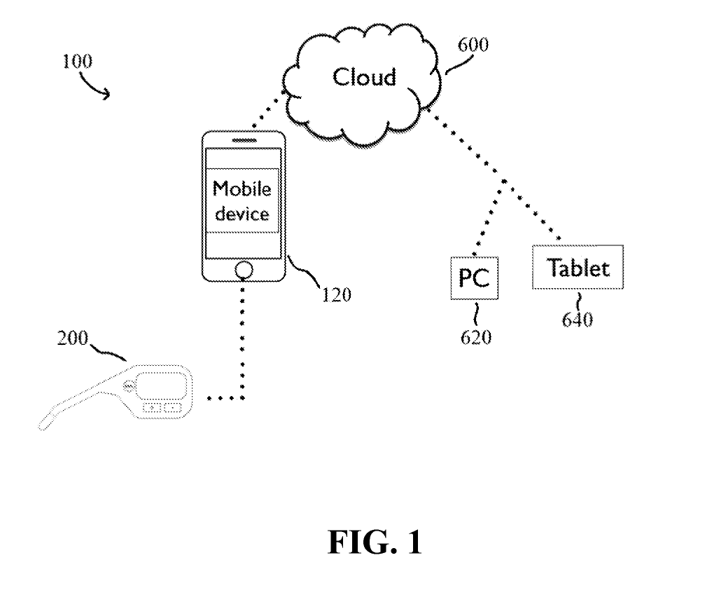

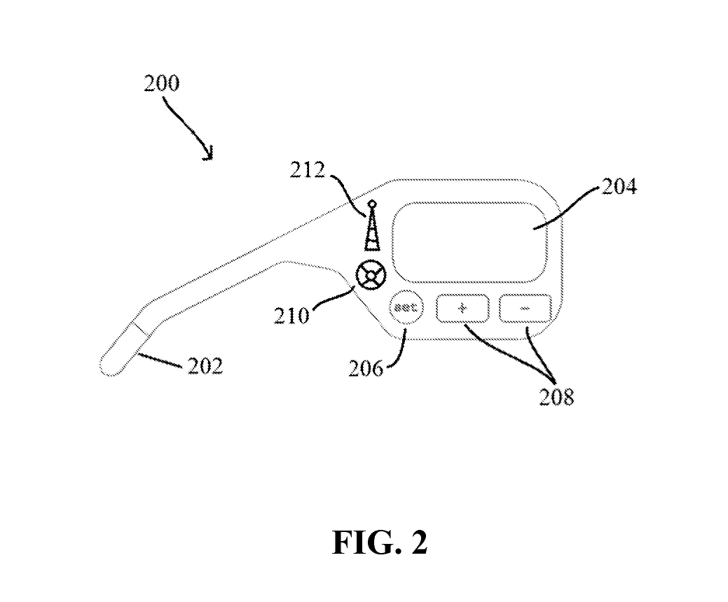

These and other objectives can be achieved by providing a system that generally includes an electronic device configured to receive inputs of basal body temperature and other indicia of fertility, determine a fertility status of the woman based thereon, and notify the woman of her fertility status. With reference now to FIG. 1, one exemplary system 100 for monitoring the fertility of a woman is depicted. As shown in FIG. 1, the system 100 generally includes a thermometer 200 in communication (e.g., wireless communication) with an electronic device 120 associated with a digital data processor configured to make a fertility status determination based at least partially on the data generated by the thermometer 200. In various aspects, the system can further comprise a remote digital data processor (e.g., cloud computing platform 600) and one or more additional electronic devices (e.g., personal computer 620, tablet computer 640) that enable the user and/or others (e.g., a medical professional) to access the basal body temperature data, input data, make fertility status predictions and determinations, and/or other related information such as diet and exercise recommendations for the woman whose fertility is being tracked based on the fertility status determination. The thermometer 200 can have a variety of configurations but generally includes a temperature probe 202 (e.g., thermistor) and a transmitter 212 for transmitting temperature data generated by the temperature probe 202 to the electronic device 120. Any known temperature data generating probes modified in accordance with the present teachings can be used to acquire data regarding the user's basal body temperature. For example, with reference now to FIG. 2, though the exemplary temperature probe 202 is depicted as being configured for sub-lingual placement, it will be appreciated by a person skilled in the art that any known or hereafter developed body temperature probe modified in accordance with the present teachings can be used to acquire a basal body temperature of the user. In accordance with various aspects, the temperature probe 202 can be ergonomically designed so as to be comfortable to the user and/or minimally invasive, as shown for example in FIG. 2. Further, at least a portion of the temperature probe can be constructed of a soft rubber that enables a user to bite down without experiencing tooth pain when the temperature probe 202 is disposed beneath the user's tongue.

With reference again to FIG. 1, the transmitter 212 for communicating with the electronic device 120 can be any transmitter known in the art or hereafter developed modified in accordance with the present teachings. In the exemplary thermometer 200, for example, the transmitter 212 can transmit the temperature data to the electronic device 120 using radio frequency signals or systems (e.g., 900 MHz, 1.4 GHz, and 5.6 GHz communication systems). It will further be appreciated that in certain embodiments, the transmitter 212 can be configured to wirelessly communicate with the electronic device 120 via Wi-Fi (e.g., a 802.11 protocol), Bluetooth®, infrared, GSM, GSM plus EDGE, CDMA, quadband, and other cellular protocols, VOIP, or any other suitable protocol.

In various aspects, the exemplary thermometer 200 of FIG. 1 can additionally include a memory module (not shown) that can store the temperature data as well as the date and time of the temperature reading for later transmission to the user's electronic device 120. In some embodiments, for example, the memory module can store the temperature data and the associated time and date of at least the past 100 temperature readings. Alternatively or in addition to the wireless transmission module discussed above, the thermometer 200 can include a port for connecting to a USB cable, for example, for recharging a battery of the thermometer 200 and/or for retrieving temperature data generated by the temperature probe 202 and stored within the memory module. By way of example, a USB cable or thumb drive can be coupled to the port such that the data generated by the temperature probe 202 can be downloaded from the memory and/or transmitted directly from the electronic device 120 via a wired transmission.

With reference again to FIG. 2, the thermometer 200 can additionally include a variety of other features including a display 204 and/or an audio output device 210. The display 204, for example, can output to the user the most recent temperature reading and/or prompt the user to perform a certain action (e.g., take a temperature reading). Similarly, the audio output device 210 can indicate to the user that a temperature reading has been taken (e.g., with a double beep) and/or prompt the user to take a temperature reading (e.g., with the sound of an alarm clock).

Additionally, as shown in FIG. 2, the thermometer 200 can optionally include a menu button 206 and user interface buttons 208 to allow the user to manipulate the thermometer 200 or its display 204. For example, the user can activate the thermometer (e.g., turn on), force the transmission of data to the electronic device 120, scroll through previous temperature data, and/or associate the data with notes regarding a particular temperature reading.

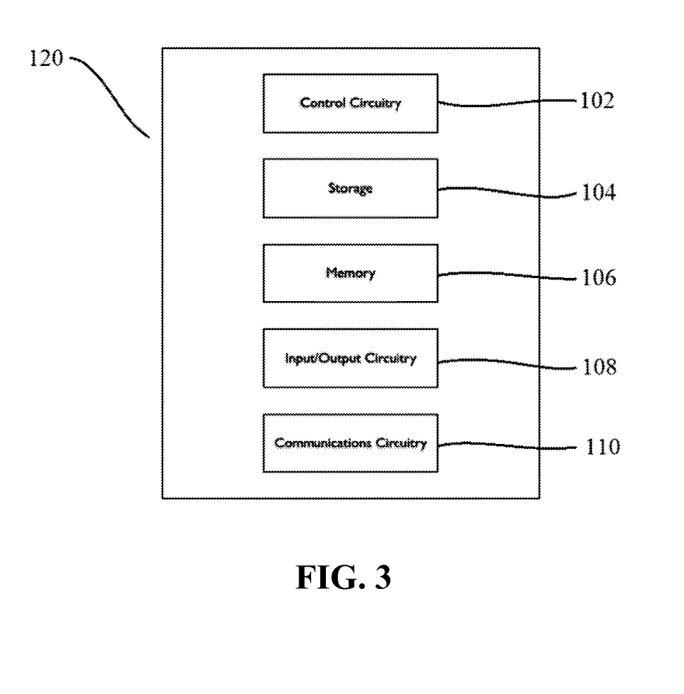

With reference now to FIG. 3, an exemplary electronic device 120 for receiving the basal body temperature data from the thermometer 200 in accordance with the present teachings is depicted in more detail. As shown in FIG. 3, the exemplary electronic device 120 can include control circuitry 102, storage 104, memory 106, input/output (“I/O”) circuitry 108, and communications circuitry 110. It will be appreciated that though the various modules are described separately based largely on their various functions, one or more of the components of electronic device 100 or functions associated therewith can be combined or omitted (e.g., storage 104 and memory 106 may be combined), or may be performed remotely through communication with one or more remote digital data processors.

Electronic device 100 can be any suitable type of electronic device presently known or hereafter developed and modified in light of the present teachings. For example, electronic device 100 can be a portable electronic device that the user may hold in his or her hand, such as a digital media player (e.g., an iPod™), a personal e-mail device or personal data assistant (e.g., a Blackberry™, PDA), a cellular telephone, a smart phone (e.g., an Android™ or iPhone™), and a tablet computer (e.g., an iPad™) Moreover, the electronic device 100 can be a larger portable electronic device, such as a laptop computer. It will further be appreciated that the electronic device 100 can be a substantially fixed electronic device, such as a desktop computer.

Control circuitry 102 can include any processing circuitry or processor operative to control the operations and performance of electronic device 100 as otherwise discussed herein. For example, control circuitry 102 can be used to run operating system applications, firmware applications, media playback applications, media editing applications, or any other application. In some embodiments, control circuitry 102 can drive a display and process inputs received from the temperature probe and/or a user interface.

Storage 104 can include, for example, one or more storage mediums including a hard-drive, solid state drive, flash memory, permanent memory such as ROM, any other suitable type of storage component, or any combination thereof Storage 104 can store, for example, application data (e.g., for implementing functions on electronic device 100), firmware, user preference information data (e.g., display preferences), authentication information, lifestyle information data (e.g., food and/or exercise preferences, food consumption and/or exercise activity data), transaction information data (e.g., information such as credit card information), wireless connection information data (e.g., information that can enable electronic device 100 to establish a wireless connection), subscription information data (e.g., information that keeps track of various media a user subscribes to), contact information data (e.g., telephone numbers and email addresses), calendar information data, and any other suitable data or any combination thereof.

Memory 106 can include cache memory, semi-permanent memory such as RAM, and/or one or more different types of memory used for temporarily storing data.

In some embodiments, memory 106 can also be used for storing data used to operate electronic device applications, or any other type of data that can be stored in storage 104. In some embodiments, memory 106 and storage 104 can be combined as a single storage medium.

I/O circuitry 108 can be operative to convert (and encode/decode, if necessary) analog signals and other signals into digital data. In some embodiments, I/O circuitry 108 can also convert digital data into any other type of signal, and vice-versa. For example, I/O circuitry 108 can receive and convert basal body temperature data, physical contact inputs (e.g., from a multi-touch screen), physical movements (e.g., from a mouse or sensor), analog audio signals (e.g., from a microphone), or any other input. The digital data can be provided to and received from control circuitry 102, storage 104, memory 106, or any other component of electronic device 100. Although I/O circuitry 108 is illustrated in FIG. 3 as a single component of electronic device 100, it will be appreciated that more than one instance of I/O circuitry 108 can be included in electronic device 100 in order to operate various input and/or output modules. Electronic device 100 can include any suitable interface or component for allowing a user to provide inputs to I/O circuitry 108. For example, electronic device 100 can include any suitable input mechanism, such as for example, a button, keypad, dial, a click wheel, or a touch screen. In some embodiments, electronic device 100 can include a capacitive sensing mechanism, or a multi-touch capacitive sensing mechanism. In some embodiments, electronic device 100 can include specialized output circuitry associated with output devices such as, for example, one or more audio outputs. The audio output can include one or more speakers (e.g., mono or stereo speakers) built into electronic device 100, or an audio component that is remotely coupled to electronic device 100.

In some embodiments, I/O circuitry 108 can include display circuitry (e.g., screen) for providing a display visible to the user. For example, the display circuitry can include a screen (e.g., an LCD screen) that is incorporated in electronics device 100. The display circuitry also can include display driver circuitry, circuitry for driving display drivers, or both. The display circuitry can be operative to display content (e.g., application screens for applications implemented on the electronic device, information regarding ongoing communications operations, information regarding incoming communications requests, or device operation screens) under the direction of control circuitry 102.

Electronic device 120 can include one more instances of communications circuitry 110 for simultaneously performing several communications operations using different communications networks, although only one is shown in FIG. 3 to avoid overcomplicating the drawing. For example, electronic device 120 can include a first instance of communications circuitry 110 for communicating with the thermometer 200 (receiving basal body temperature data transmitted from the transmitter 212) using radio signals, Bluetooth®, or otherwise, and a second instance of communications circuitry 110 for communicating over a cellular network, via Wi-Fi or using Bluetooth® to a remote digital data processor (e.g., cloud computing platform 600). Communications circuitry 110 can include any suitable communications circuitry operative to connect the electronic device 120 to the thermometer and/or a remote digital data processor via a communications network and/or to transmit data communications from electronic device 120 to the other devices. By way of example, communications circuitry 110 can be operative to interface with the thermometer 200 using any suitable communication protocol such as radio signals, Bluetooth®, or otherwise. For example, communications circuitry 110 can create a short-range communications network using a short-range communications protocol to connect to the thermometer 200. For example, communications circuitry 110 can be operative to create a local communications network using the Bluetooth® protocol to couple electronic device 120 with a Bluetooth® type transmitter within the thermometer 200. Additionally, the electronic device 120 can be configure to communicate with other devices within the communications network such as a remote digital data processor (e.g., cloud computing platform 600) using any suitable communications protocol such as, for example, Wi-Fi (e.g., a 802.11 protocol), Bluetooth®, radio frequency systems (e.g., 900 MHz, 1.4 GHz, and 5.6 GHz communication systems), infrared, GSM, GSM plus EDGE, CDMA, quadband, and other cellular protocols, VOIP, or any other suitable protocol. In some embodiments, electronic device 100 can be coupled a host device for data transfers, synching the communications device, software or firmware updates, providing performance information to a remote source (e.g., providing riding characteristics to a remove server) or performing any other suitable operation that can require electronic device 100 to be coupled to a host device. Several electronic devices 100 can be coupled to a single host device using the host device as a server. Alternatively or additionally, electronic device 100 can be coupled to several host devices (e.g., for each of the plurality of the host devices to serve as a backup for data stored in electronic device 100).

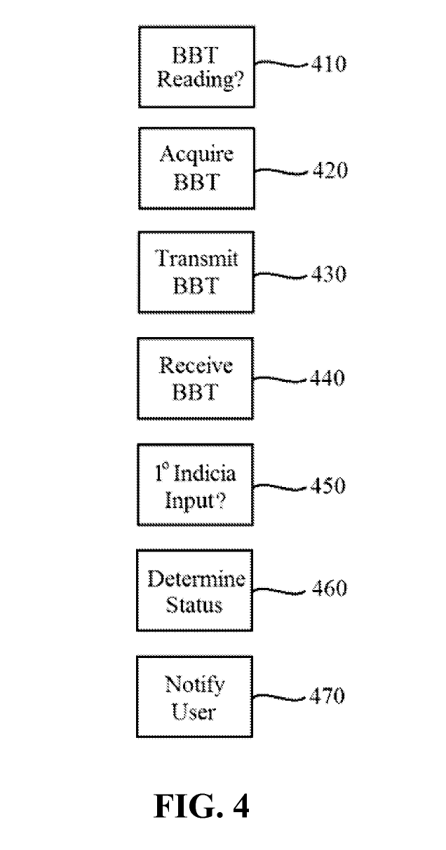

In accordance with various aspects of the present teachings, the electronic device 120 can also include an integrated software application operative to interface with the thermometer 200 in order to monitor the fertility status of a user. Various exemplary aspects of the integrated software application will now be described in conjunction with the system 100 to demonstrate by way of non-limiting example use of the system 100 in monitoring the fertility of a woman. With reference now to FIG. 4, the integrated application can prompt the user for a basal body temperature reading (step 410). By way of example, the user can be prompted for basal body temperature data via a text alert, push notification, voicemail, calendar appointment, etc. sent to the user's electronic device 120. Alternatively or additionally, the prompt can be signaled by an audio output generated by the audio output device 210 of the thermometer 200, thereby indicating to the user that basal body temperature data is requested by the application. As basal body temperature is ideally observed at the same time every day (e.g., upon waking), the application can in some embodiments access an alarm clock function of the portable electronic device such that the user is prompted to record a temperature reading immediately upon waking (e.g., immediately after the alarm clock is shut off). Alternatively or additionally, the user can be prompted to provide basal body temperature data at a fixed time every day or upon first activating the portable electronic device 120 after an extended period of inactivity or lack of motion. As the first thing that many users of portable electronic devices such as smartphones do upon waking is pick up their smartphone to check e-mail, voicemail, or other applications of the portable electronic device (e.g., a daily newsfeed), such an action after an extended period of time may indicate that the user has awoken.

As shown in step 420, whether a user is prompted to provide temperature data or otherwise, the thermometer 200 can acquire basal body temperature data of the user. For example, the user could activate the thermometer 200 and place the temperature probe 202 under her tongue until the user is notified that a temperature reading has been recorded. One or more temperature readings can be stored on the thermometer 200. As shown in step 430, the transmitter 212 can then transmit the basal body temperature data wirelessly to the electronic device 120 automatically upon generating the data or, for example, following a command by the user. Additionally or alternatively (e.g., in the case when the thermometer 200 is not within range of the electronic device 120), the basal body temperature data could be stored by the thermometer for later transmission. As shown in step 440, the portable electronic device can receive the basal body temperature data. For example, communications circuitry 110 can wirelessly receive the signal transmitted from the transmitter 212 of the thermometer 200. It will further be appreciated in light of the teachings herein that the basal body temperature data can be transmitted to the portable electronic device 120 through a wired transmission (e.g., through a USB cable, from a thumb drive that downloaded the data from the thermometer 200) or via a manual input by the user using an interface as described below for primary indicia of fertility.

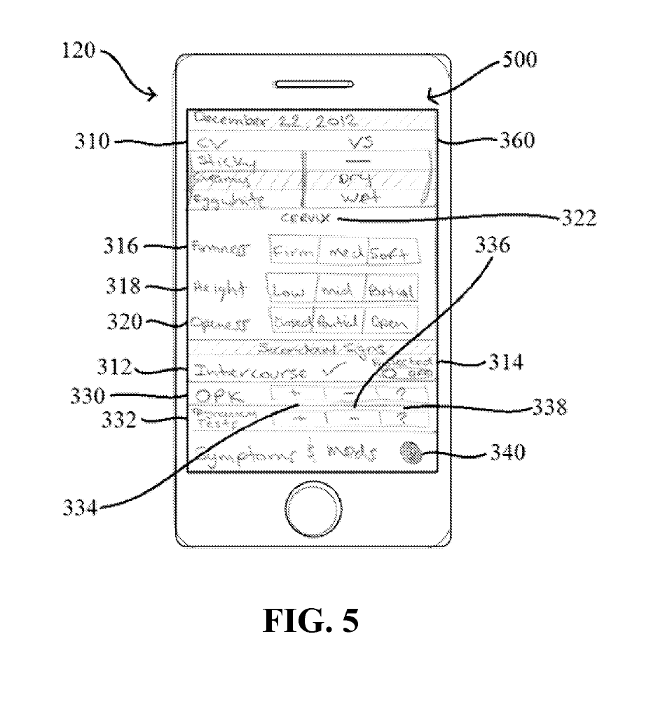

As shown in step 450, the portable electronic device can prompt the user to input data regarding one or more primary indicia of fertility as well as one or more secondary indicia of fertility. With reference now to FIG. 5, an exemplary display 500 on the portable electronic device 120 can be generated by the integrated application for prompting the user to input various primary indicia of fertility by selecting from a list of characteristics. By way of non-limiting example, the display 500 can prompt for inputs regarding characteristics of the cervical fluid 310 and vaginal sensation 360. Additionally, the display 500 can provide a list of various characteristics of the cervix, including for example, firmness 316, height 318, and openness 320. Additionally, the display can prompt the user for input regarding one or more secondary indicia of fertility, for example, when the user has intercourse 312, and whether it was protected 322. Additional non-limiting examples of secondary indicia include ovulation predictor kit results, pregnancy test results, medications taken, ferning, breast tenderness, breast fullness, and other events that may affect a woman's ovulation signs. For example, a user may input the taking of an ovulation predictor kit 330, a pregnancy test 332, or when disturbances 340 such as illness have occurred. For example, the results of an ovulation predictor kit 330 or pregnancy test 340 can be indicated by inputting a positive result 334, a negative result 336, or an inconclusive result 338. The user can also input additional information, for example, any other symptoms, diet, and exercise that can be used in monitoring fertility. For example, the integrated software application can enable the user to input medications that are being taken, events that have occurred such as travel, alcohol intake, breast self-exams, doctor's appointments, and medical tests or other procedures.

With reference again to FIG. 4, the integrated application can then determine the fertility status of the user of the electronic device 120 based on the basal body temperature data and/or the input data of the primary and secondary indicia of fertility, as shown in step 460. For example, the basal body temperature data can be charted to help determine the user's fertile and infertile phases. It will be appreciated that the fertility status determination, which can be performed using an algorithm based on the Fertility Awareness Method, for example, can be performed locally on the user's electronic device 120 or remotely. For example, the basal body temperature data and input data regarding primary and secondary indicia of fertility can be transmitted (e.g., uploaded) to the cloud computing platform 600, where the determination can be made and transmitted back to the electronic device 120.

As shown in step 470, the user can then be notified of the fertility status determined in step 460. The user could be notified in a variety of manners, for example, through a display on the electronic device 120 similar to that depicted in FIG. 5 and/or through one or more notifications delivered through the user's electronic device 120. By way of non-limiting example, the notification of the woman's fertility status can be delivered by e-mail, text message, voicemail, push notification, or pop-up notification delivered to the portable electronic device.

As shown in step 480, the system can additionally enable the user to share certain determinations or various other information used to determine the fertility status with one or more additional persons that can, in some embodiments, be running an application compatible with the application operating on the mobile device 120. By way of example, a menu can provide an option to share with a contact stored within the user's electronic device 120 certain events via text message, email, or a pop up notification. For example, the fertility status and other information can be selected for delivery to the office of the user's fertility specialist or OB/GYN (e.g., personal computer 620) via the communication network, as indicated in FIG. 1. Additionally or alternatively, the information could be delivered to the user's partner or one or more of the user's other portable electronic devices (e.g., tablet 640) so that potential reproductive activity can be considered.

Additionally or alternatively, the integrated application can enable the user to prevent unauthorized access to the application. For example, the application may require the input of a passcode so that if the portable electronic device on which the application is running is lost or stolen, the private information cannot be accessed by an unauthorized user.

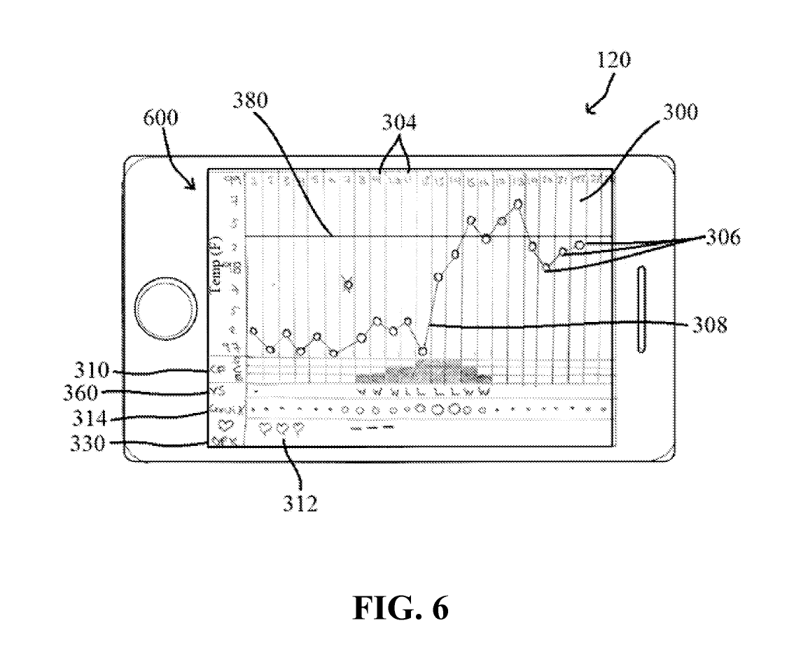

In various aspects, the integrated software application can provide additional information to the user through a display on the electronic device 120. For example, with reference now to FIG. 6, an exemplary chart 600 for display on the electronic device 120 is shown tracking various indicia of fertility when using the Fertility Awareness Method. The exemplary display 600 includes a basal body temperature chart 300 tracking the user's daily basal body temperature and displaying the temperature as a data point 306 on the date 304 the temperature is taken. As more temperatures are taken, the software application can build a plotline 308 between the temperature data points 306. The exemplary basal body temperature chart 300 can mimic the traditional Fertility Awareness Method layouts, which can make it easier for users and doctors to understand. A cover line 380, which provides an estimated threshold of the temperature upon which ovulation occurs, can be calculated based upon the temperature pattern of the plotted temperature data points 306. Additionally, below the basal body temperature chart 300, primary and secondary indicia of fertility with the

Fertility Awareness Method can be displayed. The exemplary display 600 includes, for example, indications of cervical fluid 310, vaginal sensation 360, cervical position 314, and intercourse 312.

The display prompting the user to input primary and/or secondary indicia of fertility can have a variety of configurations. By way of example, rather than the display 500 depicted in FIG. 5, the display can be in the form of a calendar that allows a user to input Fertility Awareness Method indicia. By way of example, a user may also have the ability to click on an edit button for a particular day and access various menu options. In some aspects, the integrated application can create a chart based on the input data and can display an ovulation forecast for the user's cycles. In embodiments in which users rely only on fluid and/or cervical position, and do not use basal body temperature, many users will find such a chart easier to use.

In addition to the exemplary displays and functions described above, the software application can provide additional functions for viewing, manipulating, sharing, or storing the information regarding the user's fertility status. Systems and methods in accordance with various aspects of the present teachings can, in some embodiments, increase the effectiveness of various methods for tracking fertility for the purpose of conception or contraception by providing a fertility status determination based on observable, natural phenomenon experienced by women throughout their menstrual cycles. Rather than providing a purely mathematical computation based on the average length of a woman's past cycle lengths, the methods and systems in accord with the present teachings can in some aspects enable a more accurate determination of the likelihood of fertility. For example, by providing real-time information regarding the woman's fertility status as well as relevant content throughout her cycle to her personal, portable electronic device, the integrated software application can enable improved tracking of the signs of fertility, increased adherence to the rules of the various fertility monitoring methods (e.g., Fertility Awareness Methods), and improved results, while avoiding in some cases the stress and tension resulting from worrying about compliance with the rules of the various fertility monitoring methods. Additionally, the integrated software application can, in some embodiments, enable women and partners to connect with each other and/or fertility specialists through a discussion portal to share tribulations and successes, which can be accessible to the users of the software application. Moreover, educational materials relevant to a user's particular cycle history and current condition can be delivered contemporaneously to the user's device.

For example, in some aspects, the integrated software application can provide additional information and/or literature related to the method of monitoring fertility (e.g., the Fertility Awareness Method) and/or recommendations for the user to follow based on the determined fertility status or cycle phase. By way of example, the application can recommend to the user particular foods, exercise regimes, acupressure techniques and meditation that may help in achieving pregnancy during a particular phase of the user's menstrual cycle. In some embodiments, for example, a display screen in the application can consist of a summary of the basic rules for following Fertility Awareness Method in order to help new users and clear any doubts to existing ones. Such a display can also include links to books and blogs that can help further educate users on the Fertility Awareness Method.

In some aspects, the application can provide a fertility history that can provide to the user information regarding previous cycles. In such an embodiments, a menu can be provided in which a user's previous charts will be listed and the user can select a particular chart to view without going through the calendar.

In some aspects, the application can provide ovulation forecasts that predict high likelihood of ovulation for future months, including peak day, rise in temperature, safe days for copulation without fear of reproduction, and future period.

In some aspects, the integrated software application can allow a user to back up the data contained in or generated by the application. For example, a user can choose between uploading to a cloud service, emailing one or more charts in PDF format, or backing up or restoring previous cycles.

By way of example, the portable electronic device can comprise an integrated software application configured to perform various aspects of the present teachings. In various aspects, the digital data processor can be operable to run an integrated application for interfacing with the temperature probe, for allowing the user to input one or more of the primary indicia of fertility, for determining the fertility status of the woman, and/or for notifying the user of her fertility status. In some embodiments, the integrated software application can collect and compile indicia used to track fertility and/or display the user's fertility status.

Although the preferred embodiments of the present invention have been described herein, the above description is merely illustrative. Further modification of the invention disclosed herein disclosed will occur to those skilled in the respective arts and all such modifications are deemed to be within the scope of the invention as defined by the appended claims.

What is claimed is:

1. A method of monitoring fertility in women, comprising:
receiving basal body temperature data of a user of a portable electronic device, the basal body temperature data being generated by a thermometer in communication with the portable electronic device;
receiving input data from the user of one or more primary indicia of fertility selected from the group consisting of period data, cervical fluid data, cervical softness data, cervical height data, cervical opening data, and cervical sensation data;
determining a fertility status of the user based on the basal body temperature data and the primary indicia input data; and
providing a notification to the user of her fertility status.

2. The method of claim 1, wherein the fertility status is determined by a processor of the portable electronic device.

3. The method of claim 1, further comprising transmitting the basal body temperature data and the primary indicia input data from the portable electronic device to a remote digital data processor, wherein the fertility status is determined by the remote digital data processor.

4. The method of claim 1, wherein the basal body temperature data is received using the portable electronic device.

5. The method of claim 1, wherein the primary indicia input data is received from the user using the portable electronic device.

6. The method of claim 1, wherein the notification to the user of her fertility status is provided using the portable electronic device.

7. The method of claim 1, further comprising acquiring the basal body temperature data of the user using the thermometer.

8. The method of claim 7, further comprising transmitting the basal body temperature data from the thermometer to the portable electronic device.

9. The method of claim 1, wherein providing the notification comprises outputting the fertility status to a calendar of the user.

10. The method of claim 9, wherein the calendar is displayed on the portable electronic device.

11. The method of claim 1, wherein the notification indicates that the user is one of fertile and not fertile.

12. The method of claim 11, wherein the notification comprises one of an e-mail, a text message, a voicemail, a push notification, and a pop-up notification.

13. The method of claim 12, wherein the notification is delivered to the portable electronic device.

14. The method of claim 1, further comprising prompting the user for input information regarding one or more of basal body temperature data and the primary indicia of fertility.

15. The method of claim 14, further comprising accessing a user's alarm clock on the portable electronic device.

16. The method of claim 15, wherein the user is prompted to input information regarding basal body temperature upon being woken by the alarm clock.

17. The method of claim 1, further comprising receiving input data from the user of one or more secondary indicia of fertility selected from the group consisting of ovulation predictor kits, pregnancy tests, medications, intercourse, ferning, breast fullness, breast tenderness, and disturbances.

18. The method of claim 1, further comprising providing to the user via the portable electronic device at least one of dietary and exercise information related to said fertility status.

19. A system for monitoring the fertility of a woman, comprising:
a thermometer configured to acquire basal body temperature data of a user, said thermometer having a transmitter for transmitting said basal body temperature data to a portable electronic device of the user,
said portable electronic device comprising a digital data processor configured to:
receive said basal body temperature data;
receive input data from the user of one or more primary indicia of fertility selected from the group consisting of period data, cervical fluid data, cervical softness data, cervical height data, cervical opening data, and cervical wetness data;
determine the fertility status of the user based on the basal body temperature data and the primary indicia input data; and
provide a notification to the user of her fertility status.

20. The system of claim 19, wherein the portable electronic device comprises one of a cellular phone, a smartphone, a PDA, a tablet computer, a laptop, and personal computer.

21. The system of claim 19, wherein the digital data processor is further configured to prompt the user for input information regarding one or more of basal body temperature data and the primary indicia of fertility.

22. The system of claim 19, further comprising a software application integrated into the portable electronic device.

23. The system of claim 22, wherein the software application collects and compiles indicia used to track fertility.

24. The system of claim 21, wherein the software application displays the user's fertility status.

25. The system of claim 19, wherein the digital data processor is further configured to access a calendar function of the portable electronic device and display the fertility status thereon.

26. The system of claim 19, wherein the notification comprises one of an e-mail, a text message, a voicemail, a push notification, and a pop-up notification delivered to the portable electronic device.

27. A portable electronic device for monitoring the fertility status of a woman, the portable electronic device comprising:
communications circuitry configured to interface with a thermometer for acquiring basal body temperature data of a user, said thermometer having a transmitter for transmitting said basal body temperature data,
an input component for receiving data from the user of one or more primary indicia of fertility selected from the group consisting of period data, cervical fluid data, cervical softness data, cervical height data, cervical opening data, and cervical wetness data;
a processor for determining the fertility status of the user based on the basal body temperature data and the primary indicia input data; and
an output component for notifying the user of her fertility status.

28. Machine-readable media comprising machine-readable instructions recorded thereon for:
receiving basal body temperature data of a user of a portable electronic device, the basal body temperature data being generated by a thermometer in communication with the portable electronic device;
receiving input data from the user of one or more primary indicia of fertility selected from the group consisting of period data, cervical fluid data, cervical softness data, cervical height data, cervical opening data, and cervical wetness data;
determining a fertility status of the user based on the basal body temperature data and the primary indicia input data; and
providing a notification to the user of her fertility status.
